(19) United States
(12) Patent Application Publication
(10) Pub. No.: US 2008/0139820 A1
(43) Pub. Date: Jun. 12, 2008

(54) Process for the Synthesis of Monosulfated Derivatives of Substituted Benzoxazoles

(75) Inventors: Youchu WANG, Sainte-Anne-de-Bellevue (CA); Silvio IERA, Montreal (CA); Maria PAPAMICHELAKIS, Montreal (CA)

Correspondence Address: Pepper Hamilton LLP/Wyeth, 400 Berwyn Park, 899 Cassatt Road, Berwyn, PA, 19312-1183, US

(73) Assignee: WYETH, Madison, NJ (US)

(21) Appl. No.: 11/947,061

(22) Filed: Nov. 29, 2007

Related U.S. Application Data
(60) Provisional application No. 60/867,876, filed on Nov. 30, 2006.

(51) Int. Cl. C07D 263/56

(52) U.S. Cl. 548/224

(57) ABSTRACT

The present invention provides synthetic processes for the preparation of mono-sulfated derivatives of substituted benzoxazoles, which are useful as estrogenic agents.

This application claims benefit of priority to U.S. provisional patent application Ser. No. 60/867,876 filed Nov. 30, 2006, which is hereby incorporated by reference in its entirety.

### FIELD OF THE INVENTION

This invention relates to processes for the preparation of mono-sulfated derivatives of substituted benzoxazoles, which are useful as estrogenic agents.

### BACKGROUND OF THE INVENTION

The pleiotropic effects of estrogens in mammalian tissues have been well documented, and it is now appreciated that estrogens affect many organ systems [Mendelsohn and Karas, New England Journal of Medicine 340: 1801-1811 (1999), Epperson, et al., Psychosomatic Medicine 61: 676-697 (1999), Crandall, Journal of Womens Health & Gender Based Medicine 8: 1155-1166 (1999), Monk and Brodaty, Dementia & Geriatric Cognitive Disorders 11: 1-10 (2000), Hurn and Macrae, Journal of Cerebral Blood Flow & Metabolism 20: 631-652 (2000), Calvin, Maturitas 34: 195-210 (2000), Finking, et al., Zeitschrift fur Kardiologie 89: 442-453 (2000), Brincat, Maturitas 35: 107-117 (2000), Al-Azzawi, Postgraduate Medical Journal 77: 292-304 (2001)]. Estrogens can exert effects on tissues in several ways, and the most well characterized mechanism of action is their interaction with estrogen receptors leading to alterations in gene transcription. Estrogen receptors are ligand-activated transcription factors and belong to the nuclear hormone receptor superfamily. Other members of this family include the progesterone, androgen, glucocorticoid and mineralocorticoid receptors. Upon binding ligand, these receptors dimerize and can activate gene transcription either by directly binding to specific sequences on DNA (known as response elements) or by interacting with other transcription factors (such as AP1), which in turn bind directly to specific DNA sequences [Moggs and Orphanides, EMBO Reports 2: 775-781 (2001), Hall, et al., Journal of Biological Chemistry 276: 36869-36872 (2001), McDonnell, Principles Of Molecular Regulation. p 351-361 (2000)]. A class of “coregulatory” proteins can also interact with the ligand-bound receptor and further modulate its transcriptional activity [McKenna, et al., Endocrine Reviews 20: 321-344 (1999)]. It has also been shown that estrogen receptors can suppress NFκB-mediated transcription in both a ligand-dependent and independent manner [Quaedackers, et al., Endocrinology 142: 1156-1166 (2001), Bhat, et al., Journal of Steroid Biochemistry & Molecular Biology 67: 233-240 (1998), Pelzer, et al., Biochemical & Biophysical Research Communications 286: 1153-7 (2001)].

Estrogen receptors can also be activated by phosphorylation. This phosphorylation is mediated by growth factors such as EGF and causes changes in gene transcription in the absence of ligand [Moggs and Orphanides, EMBO Reports 2: 775-781 (2001), Hall, et al., Journal of Biological Chemistry 276: 36869-36872 (2001)].

A less well-characterized means by which estrogens can affect cells is through a so-called membrane receptor. The existence of such a receptor is controversial, but it has been well documented that estrogens can elicit very rapid non-genomic responses from cells. The molecular entity responsible for transducing these effects has not been definitively isolated, but there is evidence to suggest it is at least related to the nuclear forms of the estrogen receptors [Levin, Journal of Applied Physiology 91: 1860-1867 (2001), Levin, Trends in Endocrinology & Metabolism 10: 374-377 (1999)].

Two estrogen receptors have been discovered to date. The first estrogen receptor was cloned about 15 years ago and is now referred to as ERα [Green, et al., Nature 320: 134-9 (1986)]. The second form of the estrogen receptor was found comparatively recently and is called ERβ [Kuiper, et al., Proceedings of the National Academy of Sciences of the United States of America 93: 5925-5930 (1996)]. Early work on ERβ focused on defining its affinity for a variety of ligands and indeed, some differences with ERα were seen. The tissue distribution of ERβ has been well mapped in the rodent and it is not coincident with ERα. Tissues such as the mouse and rat uterus express predominantly ERα, whereas the mouse and rat lung express predominantly ERβ [Couse, et al., Endocrinology 138: 4613-4621 (1997), Kuiper, et al., Endocrinology 138: 863-870 (1997)]. Even within the same organ, the distribution of ERα and ERβ can be compartmentalized. For example, in the mouse ovary, ERβ is highly expressed in the granulosa cells and ERα is restricted to the thecal and stromal cells [Sar and Welsch, Endocrinology 140: 963-971 (1999), Fitzpatrick, et al., Endocrinology 140: 2581-2591 (1999)]. However, there are examples where the receptors are coexpressed and there is evidence from in vitro studies that ERα and ERβ can form heterodimers [Cowley, et al., Journal of Biological Chemistry 272: 19858-19862 (1997)].

A large number of compounds have been described that either mimic or block the activity of 17β-estradiol. Compounds having roughly the same biological effects as 17β-estradiol, the most potent endogenous estrogen, are referred to as “estrogen receptor agonists”. Those which, when given in combination with 17β-estradiol, block its effects are called “estrogen receptor antagonists”. In reality there is a continuum between estrogen receptor agonist and estrogen receptor antagonist activity and indeed some compounds behave as estrogen receptor agonists in some tissues and estrogen receptor antagonists in others. These compounds with mixed activity are called selective estrogen receptor modulators (SERMS) and are therapeutically useful agents (e.g. EVISTA) [McDonnell, Journal of the Society for Gynecologic Investigation 7: S10-S15 (2000), Goldstein, et al., Human Reproduction Update 6: 212-224 (2000)]. The precise reason why the same compound can have cell-specific effects has not been elucidated, but the differences in receptor conformation and/or in the milieu of coregulatory proteins have been suggested.

It has been known for some time that estrogen receptors adopt different conformations when binding ligands. However, the consequence and subtlety of these changes has been only recently revealed. The three dimensional structures of ERα and ERβ have been solved by co-crystallization with various ligands and clearly show the repositioning of helix 12 in the presence of an estrogen receptor antagonist which sterically hinders the protein sequences required for receptor-coregulatory protein interaction [Pike, et al., Embo 18: 4608-4618 (1999), Shiau, et al., Cell 95: 927-937 (1998)]. In addition, the technique of phage display has been used to identify peptides that interact with estrogen receptors in the presence of different ligands [Paige, et al., Proceedings of the National Academy of Sciences of the United States of America 96: 3999-4004 (1999)]. For example, a peptide was identified that distinguished between ERα bound to the full estrogen receptor agonists 17β-estradiol and diethylstilbesterol. A different peptide was shown to distinguish between clomiphene bound to ERα and ERβ. These data indicate that each ligand potentially places the receptor in a unique and unpredictable conformation that is likely to have distinct biological activities.

As mentioned above, estrogens affect a panoply of biological processes. In addition, where gender differences have been described (e.g. disease frequencies, responses to challenge, etc), it is possible that the explanation involves the difference in estrogen levels between males and females.

Compounds having estrogenic activity are disclosed in U.S. Pat. No. 6,794,403, which is hereby incorporated by reference in its entirety. Given the importance of these compounds, it can be seen that a continuing need exists for new processes for their preparation. This invention is directed to these, as well as other, important ends.

### SUMMARY OF THE INVENTION

In some embodiments, the present invention provides processes for the preparation of mono-sulfated derivatives of substituted benzoxazoles, which are useful as estrogenic agents. In some embodiments, the invention provides synthetic processes comprising:

reacting (sulfating) a compound of Formula II:

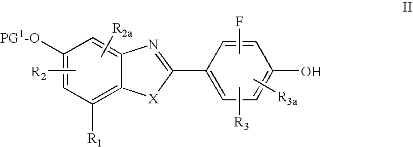

or a salt thereof, wherein:
PG¹ is a hydroxyl protecting group;
R₁ is hydrogen, hydroxyl, halogen, alkyl of 1-6 carbon atoms, trifluoroalkyl of 1-6 carbon atoms, cycloalkyl of 3-8 carbon atoms, alkoxy of 1-6 carbon atoms, trifluoroalkoxy of 1-6 carbon atoms, thioalkyl of 1-6 carbon atoms, sulfoxoalkyl of 1-6 carbon atoms, sulfonoalkyl of 1-6 carbon atoms, aryl of 6-10 carbon atoms, a 5 or 6-membered heterocyclic ring having 1 to 4 heteroatoms selected from O, N or S, —NO₂, —NR₅R₆, —N(R₅)COR₆, —CN, —CHFCN, —CF₂CN, alkynyl of 2-7 carbon atoms, or alkenyl of 2-7 carbon atoms; wherein the alkyl or alkenyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₂ and R₂ₐ are each, independently, hydrogen, hydroxyl, halogen, alkyl of 1-6 carbon atoms, alkoxy of 1-4 carbon atoms, alkenyl of 2-7 carbon atoms, or alkynyl of 2-7 carbon atoms, trifluoroalkyl of 1-6 carbon atoms, or trifluoroalkoxy of 1-6 carbon atoms; wherein the alkyl or alkenyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₃, R₃ₐ, and R₄ are each, independently, hydrogen, alkyl of 1-6 carbon atoms, alkenyl of 2-7 carbon atoms, alkynyl of 2-7 carbon atoms, halogen, alkoxy of 1-4 carbon atoms, trifluoroalkyl of 1-6 carbon atoms, or trifluoroalkoxy of 1-6 carbon atoms; wherein the alkyl or alkenyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₅, R₆ are each, independently hydrogen, alkyl of 1-6 carbon atoms, aryl of 6-10 carbon atoms;
X is O, S, or NR₇; and
R₇ is hydrogen, alkyl of 1-6 carbon atoms, aryl of 6-10 carbon atoms, —COR₅, —CO₂R₅ or —SO₂R₅,

with a sulfating reagent, for a time and under conditions sufficient to form a compound of Formula I or Ia:

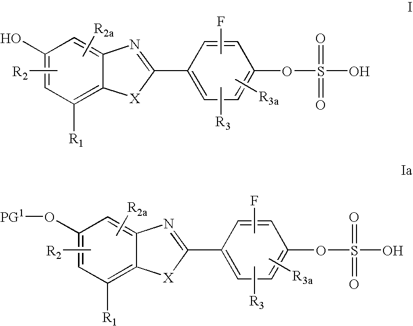

or a salt thereof.

In some further embodiments, the compound of Formula II is prepared by providing a compound of Formula IV:

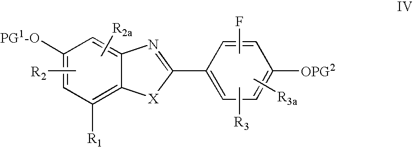

or a salt thereof;

wherein PG¹ and PG² are each independently selected hydroxyl protecting groups that can be the same or different;

and selectively removing the hydroxyl protecting group PG² to provide the compound of Formula II.

In some preferred embodiments, the compound of Formula II has the structure:

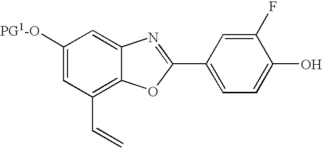

or is a salt thereof;

the compound of Formula I has the structure:

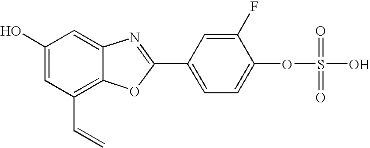

or is a salt thereof; and

the compound of Formula Ia has the structure:

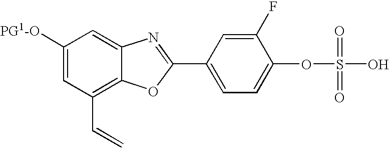

or is a salt thereof.

In some embodiments, the processes further include removing the PG¹ group of the compound of Formula Ia or the salt thereof to form the compound of Formula I or a salt thereof.

In some embodiments, the processes further include isolating a salt of the compound of Formula I or Ia, wherein the salt has the Formula Ib or Ic:

[R¹⁰—O—SO₃⁻¹]_{q}M Ib

[R¹¹—O—SO₃⁻¹]_{q}M Ic

wherein:

R¹⁰ is:

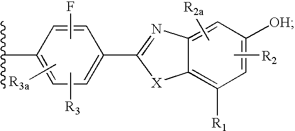

R¹¹ is:

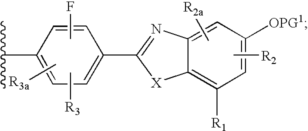

M is a Group I or II metal ion; and

q is 1 when M is Group I metal ion, or q is 2 when M is a Group II metal ion. In some embodiments, R¹⁰ has the formula R^{10a}:

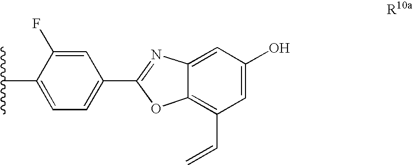

and R¹¹ has the formula R^{11a}:

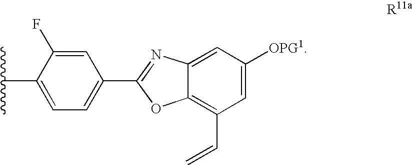

In some embodiments, M is Na+ ion.

In some preferred embodiments, the compound of Formula IV has the Formula IVa:

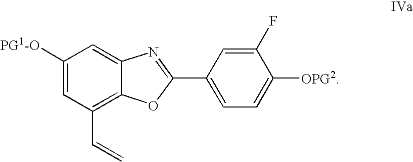

In some preferred embodiments, the compound of Formula IV is prepared by reacting a compound of Formula III:

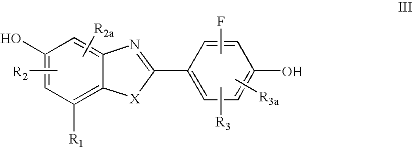

or a salt thereof, with a hydroxyl protecting group reagent for a time and under conditions sufficient to form the compound of Formula IV. In some preferred embodiments, the compound of Formula III has the Formula IIIa:

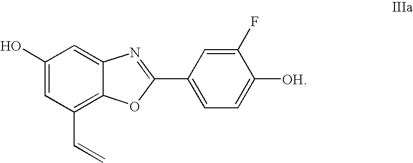

In some embodiments, PG¹ is —SiR^{a}R^{b}R^{c}; wherein R^{a}, R^{b} and R^{c} are each independently C₁₋₆ alkyl. In some preferred embodiments, PG¹ is tert-butyldimethylsilyl. In some further preferred embodiments, PG¹ and PG² are the same. In some preferred embodiments, PG¹ and PG² are each tert-butyldimethylsilyl.

### DESCRIPTION OF THE INVENTION

In some embodiments, the invention provides synthetic processes comprising:

reacting (sulfating) a compound of Formula II:

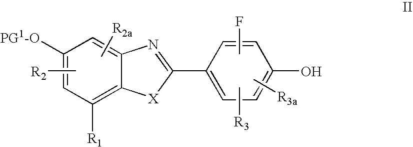

or a salt thereof, wherein:
PG¹ is a hydroxyl protecting group;
R₁ is hydrogen, hydroxyl, halogen, alkyl of 1-6 carbon atoms, trifluoroalkyl of 1-6 carbon atoms, cycloalkyl of 3-8 carbon atoms, alkoxy of 1-6 carbon atoms, trifluoroalkoxy of 1-6 carbon atoms, thioalkyl of 1-6 carbon atoms, sulfoxoalkyl of 1-6 carbon atoms, sulfonoalkyl of 1-6 carbon atoms, aryl of 6-10 carbon atoms, a 5 or 6-membered heterocyclic ring having 1 to 4 heteroatoms selected from O, N or S, —NO₂, —NR₅R₆, —N(R₅)COR₆, —CN, —CHFCN, —CF₂CN, alkynyl of 2-7 carbon atoms, or alkenyl of 2-7 carbon atoms; wherein the alkyl or alkenyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₂ and R₂ₐ are each, independently, hydrogen, hydroxyl, halogen, alkyl of 1-6 carbon atoms, alkoxy of 1-4 carbon atoms, alkenyl of 2-7 carbon atoms, or alkynyl of 2-7 carbon atoms, trifluoroalkyl of 1-6 carbon atoms, or trifluoroalkoxy of 1-6 carbon atoms; wherein the alkyl or alkenyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₃, R₃ₐ, and R₄ are each, independently, hydrogen, alkyl of 1-6 carbon atoms, alkenyl of 2-7 carbon atoms, alkynyl of 2-7 carbon atoms, halogen, alkoxy of 1-4 carbon atoms, trifluoroalkyl of 1-6 carbon atoms, or trifluoroalkoxy of 1-6 carbon atoms; wherein the alkyl or alkenyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₅, R₆ are each, independently hydrogen, alkyl of 1-6 carbon atoms, aryl of 6-10 carbon atoms;
X is O, S, or NR₇; and
R₇ is hydrogen, alkyl of 1-6 carbon atoms, aryl of 6-10 carbon atoms, —COR₅, —CO₂R₅ or —SO₂R₅,

with a sulfating reagent, for a time and under conditions sufficient to form a compound of Formula I or Ia:

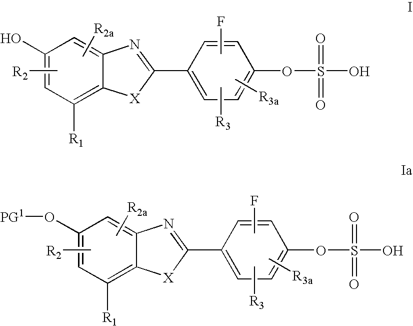

or salt thereof, or mixture thereof.

In some embodiments, the present processes are used to prepare compounds of Formula I or Ia that are substantially free of compounds of Formula X or Xa:

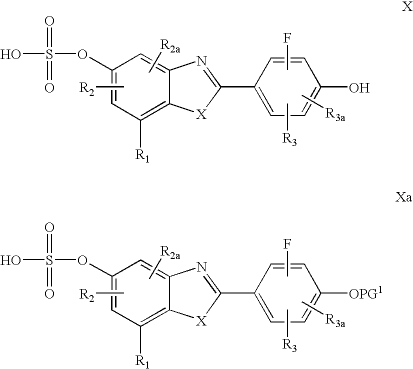

or salts thereof, wherein R₁, R₂, R₂ₐ, R₃, R₃ₐ, PG¹ and X are the same as defined in the compounds Formula I and Ia herein. As used herein, the term “substantially free of compounds of Formula X or Xa” means that no more than about 5% by weight, preferably no more than about 2% by weight, more preferably no more that about 1% by weight, and more preferably no more than about 0.5% by weight of a given sample of compound has the Formula X or Xa or a salt thereof.

A general outline of some embodiments of the processes of the present invention, wherein PG¹ and PG² are each tert-butyldimethylsilyl (TBS), is provided in Scheme I below.

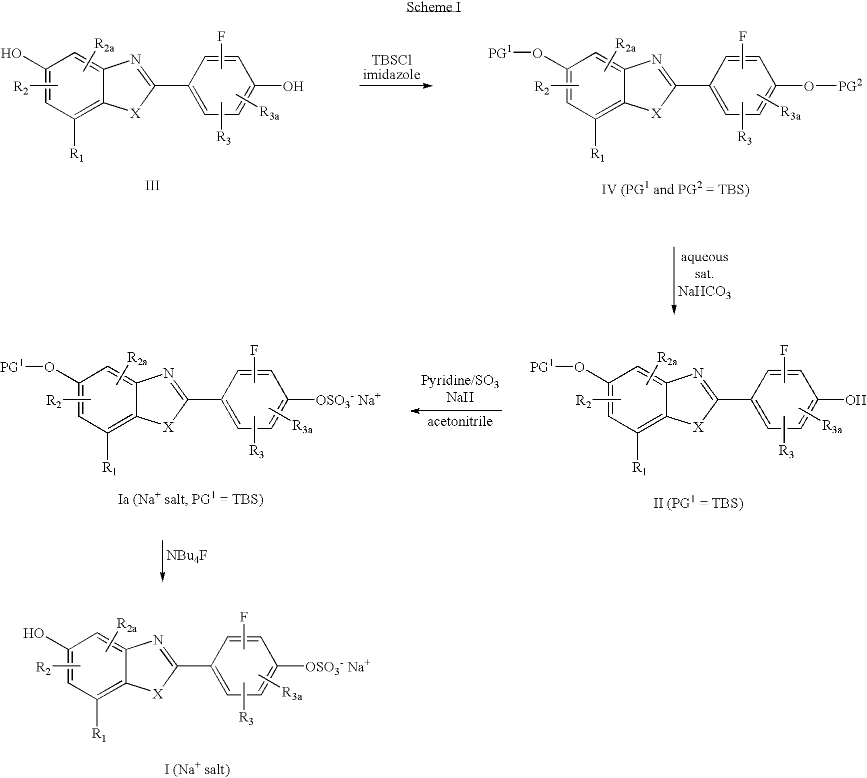

The preparation of compounds of Formula III is described in U.S. Pat. No. 6,794,403, hereby incorporated by reference in its entirety.

In some preferred embodiments, the compound of Formula II has the structure:

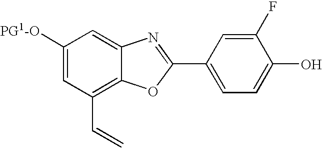

or is a salt thereof;

the compound of Formula I has the structure:

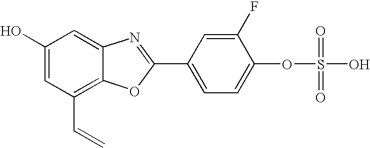

or is a salt thereof; and

the compound of Formula Ia has the structure:

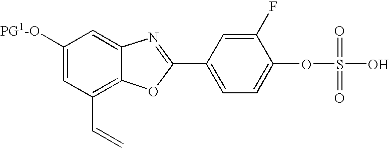

or is a salt thereof.

In some preferred embodiments, the compound of formula IV has the structure

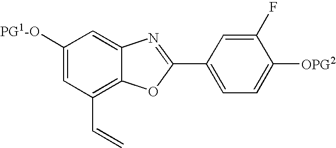

and

the compound of Formula III has the structure

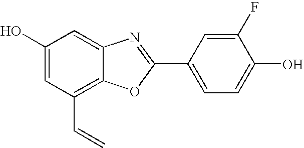

or is a salt thereof.

As can be seen in Scheme I, the starting material of Formula III has two reactive hydroxyl groups and the present invention surprisingly provides a convenient route for the preparation of the mono-sulfated product of Formula I which is substantially free of di-sulfated by-product or of the product of Formula X or Xa above (mono-sulfated at the fused ring system hydroxyl group) or their salts. The preparation of the compound of Formula I presents particular problems since we have found that the phenolic hydroxy group is relatively more acidic than the fused ring system hydroxy group. Attempts to selectively react the compound of Formula I with a protective group tend therefore preferentially to protect the phenolic hydroxyl group and lead to the sulfation of the benzooxazoyl hydroxy group rather than the desired phenolic hydroxy group. In some embodiments the present invention seeks to overcome this problem by protecting both phenolic hydroxyls to provide a compound Formula IV. Surprisingly, selective deprotection may then be used to provide the desired compound of Formula I or salt thereof. In some embodiments, compounds of Formula IV or salts thereof prepared by the present processes are substantially free of mono-protected products of the compounds of compounds of Formula II, such as compounds of Formula II or salts thereof or a mono-protected product wherein the protecting group occurs on the hydroxyl group of the phenyl ring bearing the fluoro atom. As used herein, the term “substantially free of compounds of mono-protected products of the compounds of compounds of Formula II” means that no more than about 5% by weight, preferably no more than about 2% by weight, more preferably no more that about 1% by weight, and more preferably no more than about 0.5% by weight of a given sample of compound has any mono-protected products of the compounds of compounds of Formula II, such as a compound of Formula II or salt thereof, or a mono-protected product wherein the protecting group occurs on the hydroxyl group of the phenyl ring bearing the fluoro atom. Protecting group PG¹ protects the hydroxyl group attached to the fused phenyl ring, e.g., the benzoxazole hydroxyl where X=O, and protecting group PG² protects the phenyl hydroxyl group. In some embodiments, PG¹ and PG² are the same. In embodiments where PG¹ and PG² are the same, the protecting groups are conveniently added by reacting the compound of Formula III with a hydroxyl protecting group reagent, which in some embodiments has the structure PG¹-Q, where PG¹ is a protecting group, and Q is a leaving group that is displaced by the oxygen atom of the hydroxyl to be protected. In some further embodiments, PG¹ and PG² are the same: —SiR^{a}R^{b}R^{c} wherein R^{a}, R^{b} and R^{c} are each independently C₁₋₆ alkyl.

Although Scheme I shows one preferred embodiment wherein PG¹ and PG² are the same (i.e., TBS), the protecting groups also can be different from each other. In such embodiments, two protecting group reagents would be employed serially under conditions wherein the first protecting groups reagent can react preferentially with one of the two hydroxyls. Without wishing to be bound by a particular theory, it is believed that the phenyl hydroxyl is more acidic, and its corresponding phenoxide ion less nucleophilic, than the hydroxyl and corresponding phenoxide attached to the 5-position of the fused phenyl ring (e.g., the benzoxazole hydroxyl where X=O). Accordingly, it is believed that in the presence of one equivalent or less of a strong base, for example an alkoxide or hydride ion, the phenoxide ion generated from the phenyl hydroxyl can be made to react selectively with a first protecting group reagent. Then a second protecting group reagent can be reacted with the remaining hydroxyl, preferably in the presence of a base.

The resulting compound of Formula IV is then selectively deprotected by removal of protecting group PG², whilst retaining protective group PG¹, to afford a compound of Formula II or salt thereof. The compound of Formula II or salt thereof is then reacted with a sulfating reagent to provide the sulfate compound of Formula I or Ia or salt thereof or a mixture thereof. In embodiments where the product contains a compound of Formula Ia or a salt thereof, the PG¹ protecting group of the compound of Formula Ia or salt thereof is then removed to yield the compound of Formula I, or a salt thereof.

Suitable hydroxyl protecting groups include those having the structure —SiR^{a}R^{b}R^{c} wherein R^{a}, R^{b} and R^{c} are each independently C₁₋₆ alkyl. One preferred hydroxyl protecting group is tert-butyldimethylsilyl (TBS), which can be attached to one or both hydroxyls of the compound of Formula III by reaction with the hydroxyl protecting group reagent tert-butyldimethylsilyl chloride. In some embodiments, PG¹ and PG² are the same. In some such embodiments, the hydroxyl protecting group reagent, for example tert-butyldimethylsilyl chloride, is employed in an amount that is at least about two molar equivalents, preferably about 3 or more molar equivalents relative to that of the compound of Formula III. Other suitable hydroxyl protecting groups and hydroxyl protecting group reagents are disclosed in Greene and Wuts, Protective Groups in Organic Synthesis, 2d ed, John Wiley & Sons, New York, 1991, the disclosure of which is hereby incorporated by reference in its entirety.

Typically, the reaction of the compound of Formula III and the hydroxyl protecting group reagent is performed in a solvent system, that can be a single solvent, or a mixture of solvents. A wide variety of solvents can be employed, including polar organic solvents, preferably polar aprotic organic solvents—i.e., organic solvents that are not readily deprotonated in the presence of a strongly basic reactant. Suitable aprotic solvents can include, by way of example and without limitation, hydrocarbons, alkylnitriles, dimethylformamide (DMF), dimethylsulfoxide (DMSO), dimethylacetamide (DMAC), N-methylpyrrolidinone (NMP), ethyl formate, N,N-dimethylpropionamide, dimethoxymethane, and many ether solvents including tetrahydrofuran (THF), 1,3-dioxane, 1,4-dioxane, furan, diethyl ether, diisopropyl ether, dibutyl ether, ethylene glycol dimethyl ether, ethylene glycol diethyl ether, diethylene glycol dimethyl ether, diethylene glycol diethyl ether, triethylene glycol dimethyl ether, anisole, and t-butyl methyl ether. In some embodiments, the reaction is performed in a solvent system that includes or consists of DMF.

Typically, the compound of Formula III is dissolved in a solvent system at a suitable temperature (for example room temperature), and the hydroxyl protecting group reagent is added. Preferably, a base is also added to the reaction mixture. Suitable bases include amines, hydrides such as sodium hydride and potassium hydride, and alkoxides such as potassium t-butoxide, and sodium t-butoxide. Suitable amines used as the base include acyclic amines such as alkylamines (for example, trialkylamines including triethylamine and trimethylamine), dimethylphenylamine and dimethylbenzylamine; cyclic amines (for example, pyrrolidine, piperidine, 1-methylpyrrolidine, and 1-methylpiperidine); and aromatic amines (which have one or more nitrogen atoms as ring-forming atoms of the aromatic ring, for example, imidazole, 1-methyl-imidazole, pyridine, and pyrimidine). In some embodiments, the base includes a tertiary amine (for example, triethylamine, trimethylamine, 1-methylpyrrolidine, 1-methylpiperidine, 1-methyl-imidazole, pyridine, and pyrimidine). In some embodiments, the base includes an aromatic amine, for example, imidazole, 1-methyl-imidazole pyridine, and pyrimidine. The progress of the reaction, which is typically complete in about 2 hours, can be monitored by a variety of techniques, for example by chromatographic techniques such as thin layer chromatography (TLC). In some embodiments, silyl groups (—SiR^{a}R^{b}R^{c} wherein R^{a}, R^{b} and R^{c} are each independently C₁₋₆ alkyl, for example tert-butyldimethylsilyl) are used as the hydroxyl protecting groups (PG¹ and PG²). In some such embodiments, the yield of the compound of Formula IV is greater than 80%, 85%, 88%, 92%, 95%, 98%, or 99%. In some such embodiments, the yield of the compound of Formula IV is quantitative.

The compound of Formula IV can be collected by standard workup techniques. However, in some embodiments, the compound of Formula IV is not isolated, but is rather selectively deprotected in situ by contacting the reaction mixture from the first step described in Scheme 1 above with an inorganic base such as aqueous bicarbonate ion, cabonate ion, aqueous hydroxyl ion, or an organic base such as alkylamines, or a fluoride salt, for example an tetraalkylammonium fluoride salt such as tetrabutylammonium fluoride (TBAF). In some embodiments, an aqueous solution of an inorganic base such as aqueous bicarbonate ion, cabonate ion, or aqueous hydroxyl ion is used in the selective deprotection of the compound of Formula IV or salt thereof to afford the compound Formula II or salt thereof. Typically, the deprotection reaction is complete after about 2 days. When the reaction is complete, the mono-protected compound of Formula II, or salt thereof, can be isolated form the reaction mixture by standard work-up procedures, for example by acidification of the reaction mixture to adjust the pH to about pH 4-7, removal of solvent, and chromatography, for example by flash chromatography over silica. In some embodiments, PG¹ and PG² of the compound of Formula IV are the same: both are silyl groups (—SiR^{a}R^{b}R^{c} wherein R^{a}, R^{b} and R^{c} are each independently C₁₋₆ alkyl for example tert-butyldimethylsilyl). In some embodiments, the yield of the compound of Formula II is greater than 50%, 55%, 60%, 65%, 75%, 80%, or 85%. In some embodiments, the yield of the compound of Formula II is greater than 75%, 80%, 85%, or 90%.

As seen in Scheme I above, the mono-protected compound of Formula II is then reacted with a sulfating reagent to produce a compound of Formula Ia, or a salt thereof. In some embodiments, during the workup of isolating the produced compound of Formula Ia or a salt thereof from the reaction mixture, the workup conditions are sufficient to remove the protecting group —PG¹ of the compound of Formula Ia or a salt thereof to afford the compound of Formula I or a salt thereof. In some other embodiments where workup conditions are not sufficient to remove the hydroxyl protecting group —PG¹ of the compound of Formula Ia or the salt thereof, the processes of the invention include the further step of removing the hydroxyl protecting group —PG¹ of the compound of Formula Ia or a salt thereof to afford the compound of Formula I or a salt thereof. In some embodiments, the sulfating reagent is a complex of sulfur trioxide and an amide, for example, a complex of sulfur trioxide and N,N-dimethylformamide. In some embodiments, the sulfating reagent is a complex of sulfur trioxide and an amine, for example a tertiary amine [including acyclic amines (for example, trimethylamine, triethylamine, dimethylphenylamine and dimethylbenzylamine), cyclic amines (for example, 1-methylpyrrolidine and 1-methylpiperidine) and aromatic amines which have one or more nitrogen atoms as ring-forming atoms of the aromatic ring, for example, 1-methylimidazole, pyridine and pyrimidine]. In some embodiments, the sulfating reagent is a complex of sulfur trioxide and a tertiary amine (for example, a complex of sulfur trioxide and pyridine, a complex of sulfur trioxide and trimethylamine, or a complex of sulfur trioxide and triethylamine). In some embodiments, the sulfating reagent is a complex of sulfur trioxide and aromatic amine (such as pyridine, pyrimidine, and 1-methyl-imidazole). In some embodiments, the sulfating reagent is a sulfur trioxide/pyridine complex. Other complexes of sulfur trioxide and a tertiary amine, for example, sulfur trioxide and trimethylamine complex or sulfur trioxide and triethylamine complex, can also be used as sulfating reagents. Generally, the sulfating reagent is employed in molar excess relative to the amount of compound of Formula II or salt thereof. For example, the ratio of the sulfating reagent to the compound of Formula II or the salt thereof can be a value of between about 1 and about 2, for example about 1.4 to about 1.6.

In some embodiments, the reaction of the compound of Formula II and the sulfating reagent is performed in the presence of a base. Suitable bases include hydride ion (generated from, e.g., NaH), hydroxides (such as sodium hydroxide or potassium hydroxide), and alkyl alkoxides (such as sodium ethoxide, potassium t-butoxide, and sodium t-butoxide). Accordingly, in some embodiments, the sulfating reagent is added to a solution of the compound of Formula II and the base. Generally, the sulfating reagent is employed in an amount of about 0.7 equivalent or more relative to the amount of compound of Formula II or salt thereof, preferably about 1 equivalent or more relative to the amount of compound of Formula II or salt thereof, for example about 2 equivalents or more relative to the amount of compound of Formula II or salt thereof, or about 3 or more equivalents relative to the amount of compound of Formula II or salt thereof.

Typically, the reaction of the compound of Formula II and the sulfating reagent is performed in a solvent system, that can be a single solvent, or a mixture of solvents. A wide variety of suitable solvents can be employed, including polar organic solvents, preferably polar aprotic organic solvents, including those describe above. In some embodiments, the reaction is performed in a solvent system that includes or consists of acetonitrile. In some embodiments, the yield of the compound of Formula Ia or the salt thereof is greater than 50%, 55%, 60%, 65%, 75%, 80%, or 85%. In some embodiments, the yield of the compound of Formula Ia or salt thereof is greater than 75%, 80%, 85%, 90%, or 95%.

The reaction of the compound of Formula II and the sulfating reagent is performed at convenient temperature, for example from about 20° C. to about 60° C., preferably at from about 40° C. to about 50° C. Typically, the compound of Formula II is dissolved in solvent, and the sulfating agent is added slowly. The progress of the reaction can be monitored by a variety of techniques, for example by chromatographic techniques such as thin layer chromatography. The reaction between the compound of Formula II and the sulfating reagent is typically complete after about 8 hours to about 2 days. In some embodiments, when the reaction between the compound of Formula II and the sulfating reagent is complete, unreacted base is quenched, and the compound of Formula I or Ia is isolated and obtained as the sulfate salt. In some embodiments, the salt has the Formula Ib or Ic:

[R¹⁰—O—SO₃⁻¹]_{q}M Ib

[R¹¹—O—SO₃⁻¹]_{q}M Ic

wherein:

R¹⁰ is:

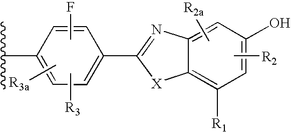

### and R¹¹ is:

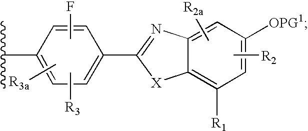

M is a Group I or II metal ion; and

q is 1 when M is Group I metal ion, or q is 2 when M is a Group II metal ion.

In some preferred embodiments, R¹⁰ has the Formula R^{10a}:

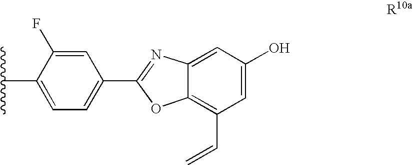

and R¹¹ has the formula R^{11a}:

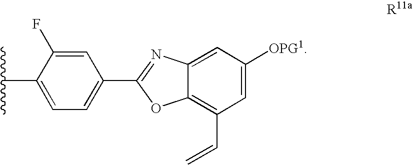

In some such embodiments, M is Na⁺ ion or K⁺ ion. In some further embodiments, M is Na⁺ ion.

The salt can be isolated from the reaction mixture by applying one or more standard techniques, for example distillation; distillation under reduced pressure; distillation further facilitated by adding a co-solvent; distillation under reduced pressure further facilitated by adding a co-solvent; filtration; evaporation of solvent followed by chromatography; or triturating the salt with an organic solvent system, for example one or more polar organic solvents. The salt can be isolated in relatively crude or in more pure form, depending upon the extent of purification. For example, in embodiments, wherein the work-up procedure does not also remove the protecting group PG¹ of the salt of the compound of Formula Ia, the salt can be isolated by treating the reaction mixture with water to quench the base, filtering and evaporating solvent to give a crude product, which can then be used as is in the subsequent deprotection step, or further purified by, for example, one or more of the foregoing techniques, such as silica chromatography.

In some embodiments where workup conditions are not sufficient to remove the hydroxyl protecting group —PG¹ of the salt of the compound of Formula Ia, the processes of the invention include the further step of removing the hydroxyl protecting group. Choice of conditions effective to remove the protecting group will vary depending on the specific protecting group employed. In some embodiments, where the hydroxyl protecting group is tert-butyldimethylsilyl (TBS), the TBS group can be removed by reaction with a fluoride salt, for example an tetraalkylammonium fluoride salt, such as tetrabutylammonium fluoride (TBAF), in a solvent, for example any of those described above, such as tetrahydrofuran. In some embodiments, the yield of the compound of Formula I or salt thereof from the compound of Formula Ia or salt thereof is greater than 60%, 65%, 75%, 80%, 85%, 88%, 90%, 92%, 95%, 98%, or 99%. In some embodiments, the yield of the compound of Formula I or salt thereof is greater than 90%, 92%, 95%, 98%, or 99%. In some embodiments, the yield of the compound of Formula I or salt thereof is quantitative.

According to a further aspect, the present invention provides a process for selectively mono-sulfating a compound of formula III to form a compound of Formula I or salt thereof, which process comprising:

a) as herein described reacting a compound of Formula III:

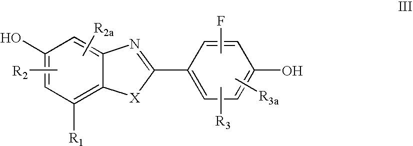

or salt thereof in an organic solvent system
with a first hydroxyl protecting group reagent PG¹-Q¹ and a second hydroxyl protecting group reagent PG²-Q² wherein Q¹ is a leaving group and Q² is a leaving group, to form the compound of Formula IV:

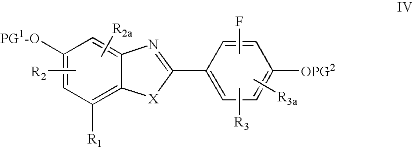

or a salt thereof;
b) as herein described contacting the compound of formula IV or a salt thereof with a base to selectively remove the hydroxyl protecting group PG² and form a compound of Formula II

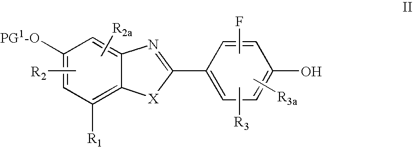

or salt thereof;
c) as herein described reacting the compound of Formula II or salt thereof with a sulfating reagent to form a compound of Formula I or Ia

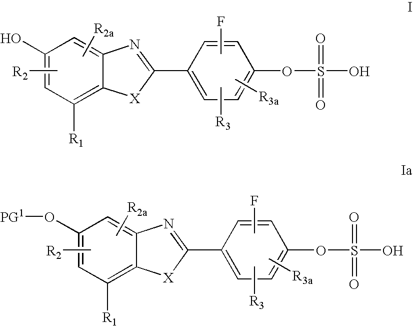

a salt thereof or a mixture thereof; and
d) as herein described removing the group PG¹ of the compound of Formula Ia if present to form the compound of formula I or salt thereof.

In some embodiments, It is unnecessary to isolate the product IV of step a) prior to step b).

Preferably, following step c) unreacted base is quenched and a salt of the compound of Formula I or Formula Ia is isolated prior to step d) as herein described, wherein the salt has the Formula Ib or Ic:

[R¹⁰—O—SO₃⁻¹]_{q}M Ia

[R¹¹—O—SO₃⁻¹]_{q}M Ic.

It is appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

The term “alkyl” employed alone, is defined herein as, unless otherwise stated, either a straight-chain or branched saturated hydrocarbon moiety. In some embodiments, the alkyl moiety contains 1 to 18, 1 to 12, 1 to 10, 1 to 8, 1 to 6, or 1 to 4 carbon atoms. Examples of saturated hydrocarbon alkyl moieties include, but are not limited to, chemical groups such as methyl, ethyl, n-propyl, isopropyl, n-butyl, tert-butyl, isobutyl, sec-butyl; higher homologs such as n-pentyl, n-hexyl, n-heptyl, n-octyl, and the like.

The term “alkylenyl” refers to a bivalent straight-chained or branched alkyl group.

As used herein, “alkenyl” refers to an alkyl group having one or more carbon-carbon double bonds. Nonlimiting examples of alkenyl groups include ethenyl, propenyl, and the like.

As used herein, “alkynyl” refers to an alkyl group having one or more carbon-carbon triple bonds. Nonlimiting examples of alkynyl groups include ethynyl, propynyl, and the like.

The term “alkoxy”, employed alone or in combination with other terms, is defined herein as, unless otherwise stated, —O-alkyl. Examples of alkoxy moieties include, but are not limited to, chemical groups such as methoxy, ethoxy, isopropoxy, sec-butoxy, tert-butoxy, and the like.

The term “cycloalkyl”, employed alone or in combination with other terms, is defined herein as, unless otherwise stated, a monocyclic, bicyclic, tricyclic, fused, bridged, or spiro monovalent non-aromatic hydrocarbon moiety of 3-18 or 3-7 carbon atoms. Also included in the definition of cycloalkyl are moieties that have one or more aromatic rings fused (i.e., having a bond in common with) to the nonaromatic ring. Any suitable ring position of the cycloalkyl moiety can be covalently linked to the defined chemical structure. Examples of cycloalkyl moieties include, but are not limited to, chemical groups such as cyclopropyl, cyclobutyl, cyclopentyl, cyclohexyl, cycloheptyl, norbornyl, adamantyl, spiro[4.5]decanyl, and the like.

The terms “halo” or “halogen”, employed alone or in combination with other terms, is defined herein as, unless otherwise stated, fluoro, chloro, bromo, or iodo.

As used herein, the term “reacting” refers to the bringing together of designated chemical reactants such that a chemical transformation takes place generating a compound different from any initially introduced into the system. Reacting can take place in the presence or absence of solvent.

The compounds of the present invention can contain an asymmetric atom, and some of the compounds can contain one or more asymmetric atoms or centers, which can thus give rise to optical isomers (enantiomers) and diastereomers. The present invention includes such optical isomers (enantiomers) and diastereomers (geometric isomers), as well as, the racemic and resolved, enantiomerically pure R and S stereoisomers, as well as, other mixtures of the R and S stereoisomers and pharmaceutically acceptable salts thereof. Optical isomers can be obtained in pure form by standard procedures known to those skilled in the art, and include, but are not limited to, diastereomeric salt formation, kinetic resolution, and asymmetric synthesis. It is also understood that this invention encompasses all possible regioisomers, and mixtures thereof, which can be obtained in pure form by standard separation procedures known to those skilled in the art, and include, but are not limited to, column chromatography, thin-layer chromatography, and high-performance liquid chromatography.

Compounds of the invention can also include all isotopes of atoms occurring in the intermediates or final compounds. Isotopes include those atoms having the same atomic number but different mass numbers. For example, isotopes of hydrogen include tritium and deuterium.

Compounds of the invention can also include tautomeric forms, such as keto-enol tautomers. Tautomeric forms can be in equilibrium or sterically locked into one form by appropriate substitution.

The processes described herein can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), or mass spectrometry, or by chromatography such as high performance liquid chromatography (HPLC) or thin layer chromatography.

The reactions of the processes described herein can be carried out in air or under an inert atmosphere. Typically, reactions containing reagents or products that are substantially reactive with air can be carried out using air-sensitive synthetic techniques that are well known to the skilled artisan.

Upon carrying out preparation of compounds according to the processes described herein, the usual isolation and purification operations such as concentration, filtration, extraction, solid-phase extraction, recrystallization, chromatography, and the like may be used to isolate the desired products.

The invention will be described in greater detail by way of specific examples. The following example is offered for illustrative purposes, and are not intended to limit the invention in any manner. Those of skill in the art will readily recognize a variety of noncritical parameters which can be changed or modified to yield essentially the same results.

### EXAMPLES

### Example 1

### Preparation of Sodium Sulfate mono-{4-[5-hydroxy-7-vinyl-benzooxazol-2-yl]-2-fluoro-phenyl}ester

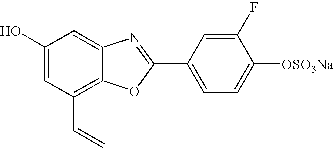

### A. Preparation of 5-(tert-butyl-dimethyl-silanyloxy)-2-[4-(tert-butyl-dimethyl-silanyloxy)-3-fluoro-phenyl]-7-vinyl-benzooxazole

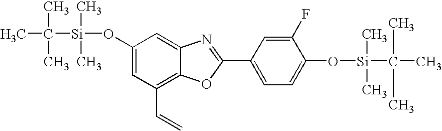

2-(3-Fluoro-4-hydroxyphenyl)-7-vinylbenzooxazol-5-ol (2 g, 7.37 mmol; prepared as described in U.S. Pat. No. 6,794,403, hereby incorporated by reference in its entirety) was dissolved in 15 mL of tetrahydrofuran (THF). Tert-butyl-dimethyl-silyl chloride (3.33 g; 22.1 mmol) and imidazole (1.51 g; 22.2 mmol) were added to the solution. The reaction mixture was then stirred for 2 hours at room temperature, and the solution was noted to become opaque white. TLC showed complete conversion of the starting material into the bis TBS-protected compound (R_{f} starting material=0.12, R_{f} bis TBS compound=0.95, EtAc/Heptane 40:60). The solution was used as is in the subsequent deprotection step.

### B. Preparation of 4-[5-(tert-butyl-dimethyl-silanyloxy)-7-vinyl-benzooxazol-2-yl]-2-fluoro-phenol

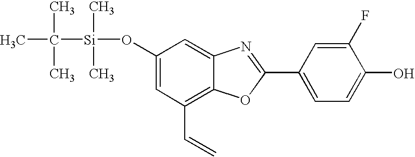

To the solution from Step A above, ethyl acetate (EtOAc, 40 mL) was added, followed by 40 mL of a saturated aqueous NaHCO₃ solution (pH=8.5), and the mixture was vigorously stirred for two days. Using a separatory funnel, the organic layer was recollected, mixed and stirred with water while cautiously adding drops of acetic acid until the pH was 6. The organic layer was recollected and evaporated (R_{f} mono TBS=0.53, EtOAc/Hept 40:60). The oily substance was purified with flash chromatography over silica (using 100% heptane, 10% EtAc/90% heptane and 100% EtOAc) to yield 3.01 g (82%) of the mono-protected compound. ¹H NMR and mass analysis were consistent with the expected structure.

### C. Preparation of sodium sulfate mono-{4-[5-(tert-butyl-dimethyl-silanyloxy)-7-vinyl-benzooxazol-2-yl]-2-fluoro-phenyl}ester

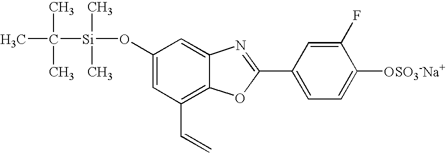

200 mg of the mono-protected compound from step B was added into a flask containing 10 mL of MeCN. The mixture was agitated and heated to 43° C. under a stream of nitrogen to dissolve the compound. In another flask, 33 mg of NaH (57% suspension in mineral oil, 1.5 eq.) was added into 5 mL MeCN and the mixture was agitated slowly under a stream of nitrogen. The solution containing the mono-protected compound was then transferred dropwise to the stirring NaH solution. The resulting mixture became yellow and transparent, and was stirred for one hour at room temperature. 0.156 g of pyridine.SO₃ was then added portionwise over a period of ½ hour, and the reaction mixture was left agitating overnight, after which TLC showed the presence of a new product. 10 drops of water were added to quench the unreacted NaH. The mixture was then filtered, and the solvents were removed with a rotary evaporator to give 262 mg of a sticky yellow oil. ¹H NMR and mass analysis were consistent with the expected structure.

### D. Preparation of sodium sulfate mono-{4-[5-hydroxy-7-vinyl-benzooxazol-2-yl]-2-fluoro-phenyl}ester

The crude yellow oil (20 mg) from step C above was dissolved in MeCN. Tetrabutylammoniun fluoride (TBAF; 1.0 M solution in THF, 0.5 mL) was added to the solution, and the yellow transparent solution turned purple-black. The mixture was stirred for two hours, and then the solvent was removed by a rotary evaporator to yield 150 mg of a purple oil.

The purification of the crude product was performed with preparative HPLC according to the following procedure.

### Conditions for Preparative HPLC:

Column: Phenomenex C₁₈ (500×100.00 mm) LUNA
Mobile phase: Solution A: Solution B=100:0 (10 mins)→(50 min)→0:100 (30 min)
Flow: 100 mL/min.
UV: 254 nm
Sample: 400 mg/60 mL in solution A
Inj: 60 mL
Temp: room temperature
Solution A: 77.2 g Ammoniumacetate; 18 L water, 1.2 L MeOH, 800 mL MeCN
Solution B: 77.2 g Ammoniumacetate; 2 L water, 10.8 L MeOH, 7.2 L MeCN

The preparative HPLC yielded two minor peaks at about 50 minutes and about 80 minutes, and a major peak at about 70 minutes that contained the sulfated product. This fraction was recollected in several 100 mL fractions. The fractions were then individually passed in HPLC to verify purity. The satisfactory fractions were added together, rotavapored to remove MeOH/MeCN and then freeze-dried. ¹H NMR and mass analysis were consistent with the expected structure. In a typical preparative HPLC procedure as used herein, the recovery rate for sodium sulfate mono-{4-[5-hydroxy-7-vinyl-benzooxazol-2-yl]-2-fluoro-phenyl}ester is about 30-50% (based on the amount of the crude product), and the purity of the final product is greater than 90%, 92%, 95%, 96%, 97%, or 98%. The purified salt is an off-white solid.

Those skilled in the art will recognize that various changes and/or modifications may be made to aspects or embodiments of this invention and that such changes and/or modifications may be made without departing from the spirit of this invention. Therefore, it is intended that the appended claims cover all such equivalent variations as will fall within the spirit and scope of this invention.

It is intended that each of the patents, applications, and printed publications, including books, mentioned in this patent document be hereby incorporated by reference in their entirety.

What is claimed is:

1. A synthetic process comprising:
reacting a compound of Formula II: 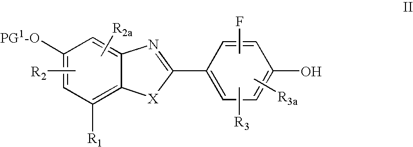
or salt thereof, wherein:
PG¹ is a hydroxyl protecting group;
R₁ is hydrogen, hydroxyl, halogen, alkyl of 1-6 carbon atoms, trifluoroalkyl of 1-6 carbon atoms, cycloalkyl of 3-8 carbon atoms, alkoxy of 1-6 carbon atoms, trifluoroalkoxy of 1-6 carbon atoms, thioalkyl of 1-6 carbon atoms, sulfoxoalkyl of 1-6 carbon atoms, sulfonoalkyl of 1-6 carbon atoms, aryl of 6-10 carbon atoms, a 5 or 6-membered heterocyclic ring having 1 to 4 heteroatoms selected from O, N or S, —NO₂, —NR₅R₆, —N(R₅)COR₆, —CN, —CHFCN, —CF₂CN, alkynyl of 2-7 carbon atoms, or alkenyl of 2-7 carbon atoms; wherein the alkyl or alkenyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₂ and R₂ₐ are each, independently, hydrogen, hydroxyl, halogen, alkyl of 1-6 carbon atoms, alkoxy of 1-4 carbon atoms, alkenyl of 2-7 carbon atoms, or alkynyl of 2-7 carbon atoms, trifluoroalkyl of 1-6 carbon atoms, or trifluoroalkoxy of 1-6 carbon atoms; wherein the alkyl or alkenyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₃, R₃ₐ, and R₄ are each, independently, hydrogen, alkyl of 1-6 carbon atoms, alkenyl of 2-7 carbon atoms, alkynyl of 2-7 carbon atoms, halogen, alkoxy of 1-4 carbon atoms, trifluoroalkyl of 1-6 carbon atoms, or trifluoroalkoxy of 1-6 carbon atoms; wherein the alkyl or alkenyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₅, R₆ are each, independently hydrogen, alkyl of 1-6 carbon atoms, aryl of 6-10 carbon atoms;
X is O, S, or NR₇; and
R₇ is hydrogen, alkyl of 1-6 carbon atoms, aryl of 6-10 carbon atoms, —COR₅, —CO₂R₅ or —SO₂R₅;
with a sulfating reagent, for a time and under conditions sufficient to form a compound of Formula I or Ia: 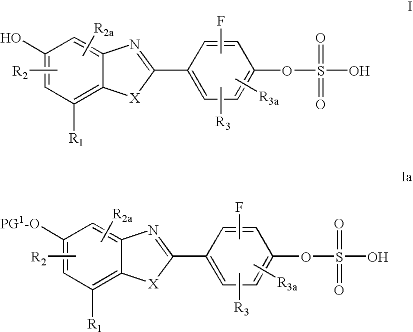
or salt thereof or mixture thereof.

2. The process of claim 1, wherein the compound of Formula II has the structure: 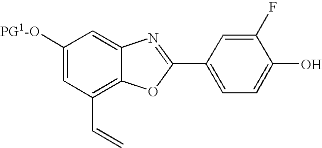
or is a salt thereof;
the compound of Formula I has the structure: 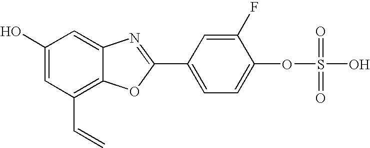
or is a salt thereof; and
the compound of Formula Ia has the structure: 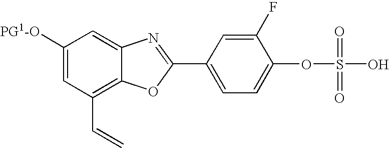
or is a salt thereof.

3. The process of claim 1, wherein PG¹ is —SiR^{a}R^{b}R^{c} and wherein R^{a}, R^{b} and R^{c} are each independently C₁₋₆ alkyl.

4. The process of claim 1, wherein PG¹ is tert-butyldimethylsilyl.

5. The process of claim 1, wherein the sulfating reagent comprises a complex of sulfur trioxide and a tertiary amine.

6. The process of claim 1, wherein the sulfating reagent comprises sulfur trioxide pyridine complex.

7. The process of claim 1, wherein the sulfating is performed in a solvent system comprising a polar, aprotic organic solvent.

8. The process of claim 1, further comprising contacting the compound of Formula I or Ia, or salt thereof, in the solvent system, with a base.

9. The process of claim 8 where the base is selected from hydride ion and an alkoxide ion.

10. The process of claim 1, further comprising isolating the compound of Formula Ia or a salt thereof.

11. The process of claim 1, further comprising removing the PG¹ group of the compound of Formula Ia or the salt thereof to form the compound of Formula I or salt thereof.

12. The process of claim 11, wherein the removing of the PG¹ group of the compound of Formula Ia or salt thereof comprises contacting the compound of Formula Ia or the salt thereof with a fluoride salt.

13. The process of claim 12, wherein the fluoride salt comprises tetrabutylammonium fluoride.

14. The process of claim 1, further comprising the step of isolating a salt of the compound of Formula I or Formula Ia, wherein the salt has the Formula Ib or Ic:
[R¹⁰—O—SO₃⁻¹]_{q}M Ib
[R¹¹—O—SO₃⁻¹]_{q}M Ic
wherein:
R¹⁰ is: 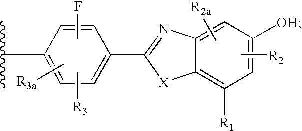
R¹¹ is: 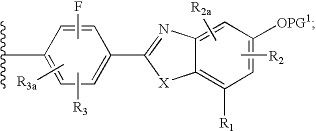
M is a Group I or II metal ion; and
q is 1 when M is Group I metal ion, or q is 2 when M is a Group II metal ion.

15. The process of claim 14, wherein R¹⁰ has the formula R^{10a}: 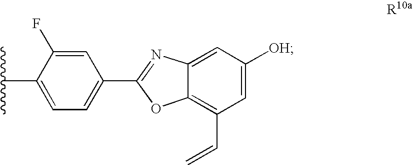
R¹¹ has the formula R^{11a}:

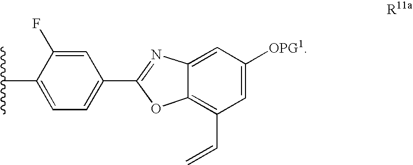

16. The process of claim 15 wherein M is Na⁺ ion.

17. The process of claim 15, wherein the isolating of the salt of Formula Ib or Ic comprises one or more of distillation, distillation under reduced pressure, distillation further facilitated by adding a co-solvent, distillation under reduced pressure further facilitated by adding a co-solvent, triturating the salt with an organic solvent system comprising a polar organic solvent, high performance liquid chromatography (HPLC) and freeze drying.

18. The process of claim 17, wherein the isolating of the salt of Formula Ib or Ic comprises HPLC.

19. The process of claim 1, further comprising:
providing a compound of Formula IV: 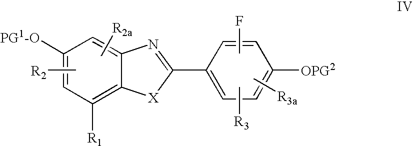
or salt thereof; wherein PG¹ and PG² are each independently selected hydroxyl protecting groups that can be the same or different;
and selectively removing the hydroxyl protecting group PG² to provide the compound of Formula II or salt thereof.

20. The process of claim 19, wherein the compound of Formula IV has the Formula IVa:

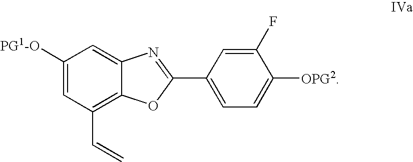

21. The process of claim 20, wherein removing the hydroxyl protecting group PG² comprises contacting the compound of Formula IV or salt thereof with base.

22. The process of claim 20, wherein removing the hydroxyl protecting group PG² comprises contacting the compound of Formula IV or salt thereof with aqueous base.

23. The process of claim 20, wherein removing the hydroxyl protecting group PG² comprises contacting the compound of Formula IV or salt thereof with aqueous bicarbonate ion.

24. The process of claim 20, wherein PG¹ and PG² are the same.

25. The process of claim 20, wherein PG¹ and PG² are each tert-butyldimethylsilyl.

26. The process of claim 19, further comprising reacting a compound of Formula III: 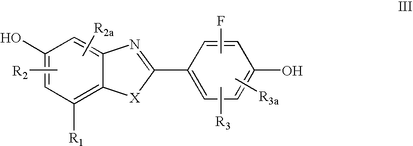
or salt thereof, with a hydroxyl protecting group reagent for a time and under conditions sufficient to form the compound of Formula IV or salt thereof.

27. The process of claim 26, wherein the compound of Formula III has Formula IIIa:

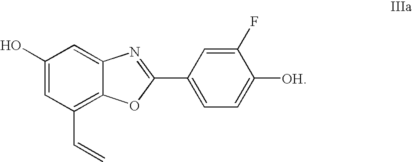

28. The process of claim 27, wherein the hydroxyl protecting group reagent is tert-butyldimethylsilyl chloride.

29. A compound of Formula IV: 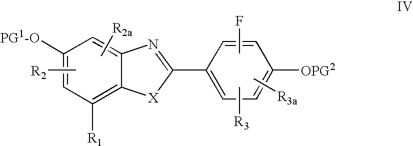
or salt thereof, wherein:
PG¹ and PG² are each independently selected hydroxyl protecting groups that can be the same or different;
R₁ is hydrogen, hydroxyl, halogen, alkyl of 1-6 carbon atoms, trifluoroalkyl of 1-6 carbon atoms, cycloalkyl of 3-8 carbon atoms, alkoxy of 1-6 carbon atoms, trifluoroalkoxy of 1-6 carbon atoms, thioalkyl of 1-6 carbon atoms, sulfoxoalkyl of 1-6 carbon atoms, sulfonoalkyl of 1-6 carbon atoms, aryl of 6-10 carbon atoms, a 5 or 6-membered heterocyclic ring having 1 to 4 heteroatoms selected from O, or S, —NO₂, —NR₅R₆, —N(R₅)COR₆, —CN, —CHFCN, —CF₂CN, alkynyl of 2-7 carbon atoms, or alkenyl of 2-7 carbon atoms; wherein the alkyl or alkenyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₂ and R₂ₐ are each, independently, hydrogen, hydroxyl, halogen, alkyl of 1-6 carbon atoms, alkoxy of 1-4 carbon atoms, alkenyl of 2-7 carbon atoms, or alkynyl of 2-7 carbon atoms, trifluoroalkyl of 1-6 carbon atoms, or trifluoroalkoxy of 1-6 carbon atoms; wherein the alkyl or alkenyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₃, R₃ₐ, and R₄ are each, independently, hydrogen, alkyl of 1-6 carbon atoms, alkenyl of 2-7 carbon atoms, alkynyl of 2-7 carbon atoms, halogen, alkoxy of 1-4 carbon atoms, trifluoroalkyl of 1-6 carbon atoms, or trifluoroalkoxy of 1-6 carbon atoms; wherein the alkyl or alkenyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₅, R₆ are each, independently hydrogen, alkyl of 1-6 carbon atoms, aryl of 6-10 carbon atoms;
X is O, S, or NR₇; and
R₇ is hydrogen, alkyl of 1-6 carbon atoms, aryl of 6-10 carbon atoms, —COR₅, —CO₂R₅ or —SO₂R₅.

30. The compound of claim 29, having the Formula IVa: [chemical structure]
